(19) United States
(12) Patent Application Publication
(10) Pub. No.: US 2024/0335611 A1
(43) Pub. Date: Oct. 10, 2024

(54) CONNECTORS FOR DUAL PLUNGER SYRINGES

(71) Applicant: CLEARSTREAM TECHNOLOGIES LIMITED, Enniscorthy (IE)

(72) Inventors: Matthew Cullen, Wicklow (IE); Dylan Beyhl, Scottsdale, AZ (US); Jordan P. Addison, Gilbert, AZ (US); Victor Bell, Dublin (IE)

(73) Assignee: CLEARSTREAM TECHNOLOGIES LIMITED, Enniscorthy (IE)

(21) Appl. No.: 18/748,973

(22) Filed: Jun. 20, 2024

Related U.S. Application Data
(63) Continuation-in-part of application No. 17/906,420, filed on Sep. 15, 2022.
(63) Continuation-in-part of application No. 18/712,812, filed on Jan. 1, 0001.
(60) Provisional application No. 62/992,191, filed on Mar. 20, 2020.
(60) Provisional application No. 63/509,355, filed on Jun. 21, 2023.

(51) Int. Cl. A61M 5/31; A61J 1/20

(52) U.S. Cl. CPC A61M 5/31; A61J 1/20

(57) ABSTRACT

A component delivery system is disclosed. The component delivery system includes an applicator including two chambers separate from one another, each chamber including at least one output port on a distal end thereof for receiving corresponding pistons. The component delivery system further includes a two-piece alignment fixture removably couplable to the applicator and a two-piece piston insert removably couplable to the pistons, the two-piece piston insert engagable with the two-piece alignment fixture to prevent removal of the pistons from the two chambers.

### CROSS-REFERENCE TO RELATED APPLICATION

The present application claims the benefit of U.S. Provisional Application No. 63/509,355, entitled CONNECTORS FOR DUAL PLUNGER SYRINGES, and filed Jun. 21, 2023, the entirety of which is hereby incorporated by reference. The present application is further a continuation-in-part of U.S. patent application Ser. No. 17/906,420, entitled MULTI-COMPONENT SEALANT DELIVERY SYSTEMS INCORPORATING QUARTER TURN CONNECTORS, filed on Sep. 15, 2022 as a National Stage Entry of PCT/US2021/023171, entitled MULTI-COMPONENT SEALANT DELIVERY SYSTEMS INCORPORATING QUARTER TURN CONNECTORS, filed on Mar. 19, 2021, which claims the benefit of U.S. Provisional Patent Application Ser. No. 62/992,191, entitled SEALANT DELIVERY SYSTEM INCLUDING SEALANT APPLICATOR DEVICE, INJECTION NEEDLE ASSEMBLY, AND COMPONENT MIXER ASSEMBLY, filed on Mar. 20, 2020, the entireties of which are hereby incorporated by reference. The present application is further a continuation-in-part of U.S. patent application Ser. No. 18/712,812, entitled DEVICES, SYSTEMS, AND METHODS FOR DELIVERY OF MULTI-COMPONENT SPACERS AND HYDRO-DISSECTION SOLUTIONS, filed on May 23, 2024 as a National Stage Entry of PCT/US2021/060570, entitled DEVICES, SYSTEMS, AND METHODS FOR DELIVERY OF MULTI-COMPONENT SPACERS AND HYDRO-DISSECTION SOLUTIONS, filed on Nov. 23, 2021, the entireties of which are hereby incorporated by reference.

### BACKGROUND

### Field

The present disclosure generally relates to component delivery systems, and more specifically, to systems and methods for preparing the same. The present disclosure further generally relates to dual plunger syringes, and more specifically, to components that aid the use of dual plunger syringes.

### Technical Background

Pneumothorax is a problematic complication of the lung biopsy procedure where air or fluid is allowed to pass into the pleural space as a result of the puncture of the parietal pleura and visceral pleura. Pneumothorax and, more so, pneumothorax requiring chest tube placement, are significant concerns for clinicians performing, and patients undergoing, percutaneous lung biopsies. The incidence of pneumothorax in patients undergoing percutaneous lung biopsy has been reported to be anywhere from 9-54%, with an average of around 15%. On average, 6.6% of all percutaneous lung biopsies result in pneumothorax requiring a chest tube to be placed, which results in an average hospital stay of 2.7 days.

Factors that increase the risk of pneumothorax include increased patient age, obstructive lung disease, increased depth of a lesion, multiple pleural passes, increased time that an access needle lies across the pleura, and traversal of a fissure. Pneumothorax may occur during or immediately after the procedure, which is why typically a CT scan of the region is performed following removal of the needle. Other, less common, complications of percutaneous lung biopsy include hemoptysis (coughing up blood), hemothorax (a type of pleural effusion in which blood accumulates in the pleural cavity), infection, and air embolism.

It has been suggested that approximately 30% of lung biopsies result in some form of pneumothorax that makes deploying a plug after the biopsy difficult or impossible. Conventional sealants are deployed through a coaxial cannula after a biopsy is performed which may be before or after a pneumothorax forms.

A number of procedures utilize dual barrel syringes for component delivery and/or removal. However, for some multi-component materials, it may be necessary to ensure that the syringes dispense fluid at a similar, or substantially the same, rate in order to ensure proper mixing of the components. In such instances, a user of a dual barrel syringe must ensure that the pistons are depressed at substantially the same time and traverse substantially the same distance. It may be difficult for a user to adequately grip or hold some syringes in order to effect such a synchronized depressing.

### SUMMARY

A component delivery system is disclosed. The component deliver system includes a dual chamber applicator including a body including a first component chamber and a second component chamber. The dual chamber applicator further includes a first piston slidably engaged with the first component chamber and a second piston slidably engaged with the second component chamber. The component delivery system further includes an alignment fixture removably coupled to the body of the dual chamber applicator and a piston insert removably couplable to the first piston and the second piston to connect the first piston to the second piston.

A component delivery system is disclosed. The component delivery system includes an applicator including two chambers separate from one another, each chamber including at least one output port on a distal end thereof for receiving corresponding pistons. The component delivery system further includes a two-piece alignment fixture removably couplable to the applicator and a two-piece piston insert removably couplable to the pistons, the two-piece piston insert engagable with the two-piece alignment fixture to prevent removal of the pistons from the two chambers.

An alignment fixture for coupling to an applicator is disclosed. The alignment fixture includes a body including a first section, a second section removably coupled to the first section in an assembled state, the body defining an opening in the assembled state to receive a plunger of the applicator therethrough, and a face including a feature to facilitate coupling of the alignment fixture to the applicator.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1A depicts a front view of an illustrative component delivery system including an applicator, a dual chamber mixing syringe, and an injection needle assembly according to one or more aspects shown and described herein;

FIG. 1B depicts a front view of an illustrative component delivery system including an applicator, an auxiliary applicator, and an injection needle assembly according to one or more aspects shown and described herein;

FIG. 2A depicts a front view of an illustrative mixing apparatus including an applicator and a dual chamber mixing syringe according to one or more aspects shown and described herein;

FIG. 2B depicts a front view of an illustrative delivery apparatus including an applicator and an injection needle assembly according to one or more aspects shown and described herein;

FIG. 3A schematically depicts a two-piece alignment fixture for use with a component delivery system according to one or more aspects shown and described herein;

FIG. 3B schematically depicts a placement of the two-piece alignment fixture of FIG. 3A around a proximal end of an applicator according to one or more aspects shown and described herein;

FIG. 3C schematically depicts the two-piece alignment fixture of FIG. 3A assembled around the proximal end of the applicator of FIG. 3B according to one or more embodiments shown and described herein;

FIG. 4A schematically depicts a two-piece alignment fixture for use with a component delivery system, according to one or more aspects shown and described herein;

FIG. 4B schematically depicts a portion of the two-piece alignment fixture of FIG. 4A according to one or more aspects shown and described herein;

FIG. 4C schematically depicts a portion of the two-piece alignment fixture of FIG. 4A coupled to a proximal end of an applicator according to one or more aspects shown and described herein;

FIG. 5 schematically depicts the two-piece alignment fixture of FIG. 4A assembled to the applicator of FIG. 4C and a two-piece piston insert according to one or more aspects shown and described herein;

FIG. 6A schematically depicts a portion of the two-piece piston insert of FIG. 4 according to one or more aspects shown and described herein;

FIG. 6B schematically depicts a frontal view of the two-piece piston insert of FIG. 6A according to one or more aspects shown and described herein;

FIG. 6C schematically depicts a perspective view of the two-piece piston insert of FIG. 6A according to one or more aspects shown and described herein;

FIG. 6D schematically depicts a frontal view of a modified two-piece piston insert according to one or more aspects shown and described herein;

FIG. 7 schematically depicts a plunger for use with a two-piece piston insert according to one or more aspects shown and described herein; and

FIG. 8 schematically depicts a two-piece piston insert partially mounted to the plunger of FIG. 7 according to one or more aspects shown and described herein.

### DETAILED DESCRIPTION

The present disclosure, in one form, is related to component delivery systems. The component delivery systems described herein include components that deliver the multicomponent material along an access path. The component delivery systems described herein include a dual chamber applicator and an injection needle assembly. The component delivery systems described herein are configured such that a user can quickly and easily grasp the applicator for the purposes of mixing and/or delivery of the materials stored, or otherwise supported, within the systems. To achieve this, the systems described herein incorporate a two-piece alignment fixture around a proximal end of the dual chamber applicator, the two-piece alignment fixture having wings and/or other features to provide a gripping or holding surface, as well as protrusions that prevent accidental removal of pistons from the dual chamber applicator. In addition, the pistons are coupled to a two-piece piston insert that integrates with each piston to provide additional grip and to ensure the pistons are depressed or pulled together.

An advantage of the present disclosure is that the various aspects described herein improve upon typical solutions that do not provide wings or other similar features for allowing a user to grip or hold while mixing a plurality of components to be delivered.

Another advantage of the present disclosure is that the two-piece piston insert aids in ensuring that both pistons are depressed or pulled at the same or substantially the same time by providing a stable and intuitive area for gripping or holding and by engaging with the pistons to maintain an alignment of the pistons.

Another advantage of the present disclosure is that the two-piece alignment fixture allows for one handed use of the dual chamber applicator to mix components.

Another advantage of the present disclosure is that the two-piece alignment fixture allows for secure and easy placement, as well as easy removal if desired.

Another advantage of the present disclosure is the combination of the two-piece alignment fixture and the two-piece piston insert engage with one another such that the two-piece alignment fixture prevents retraction/pulling of the pistons out of the dual chamber applicator (e.g., such that a user cannot accidentally pull the pistons too far).

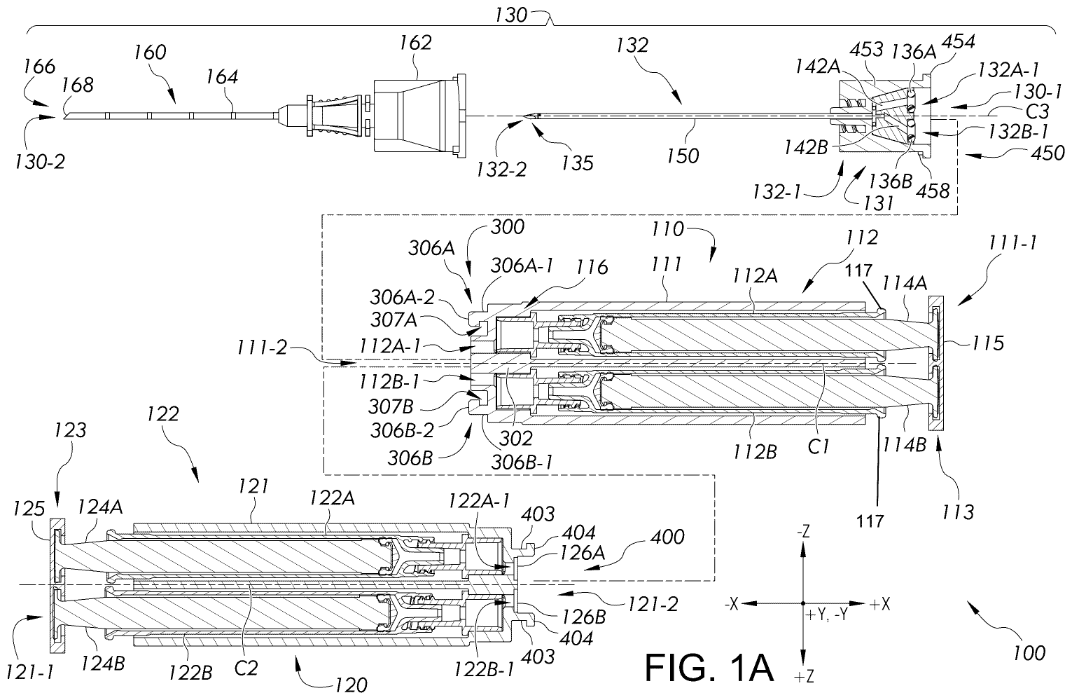

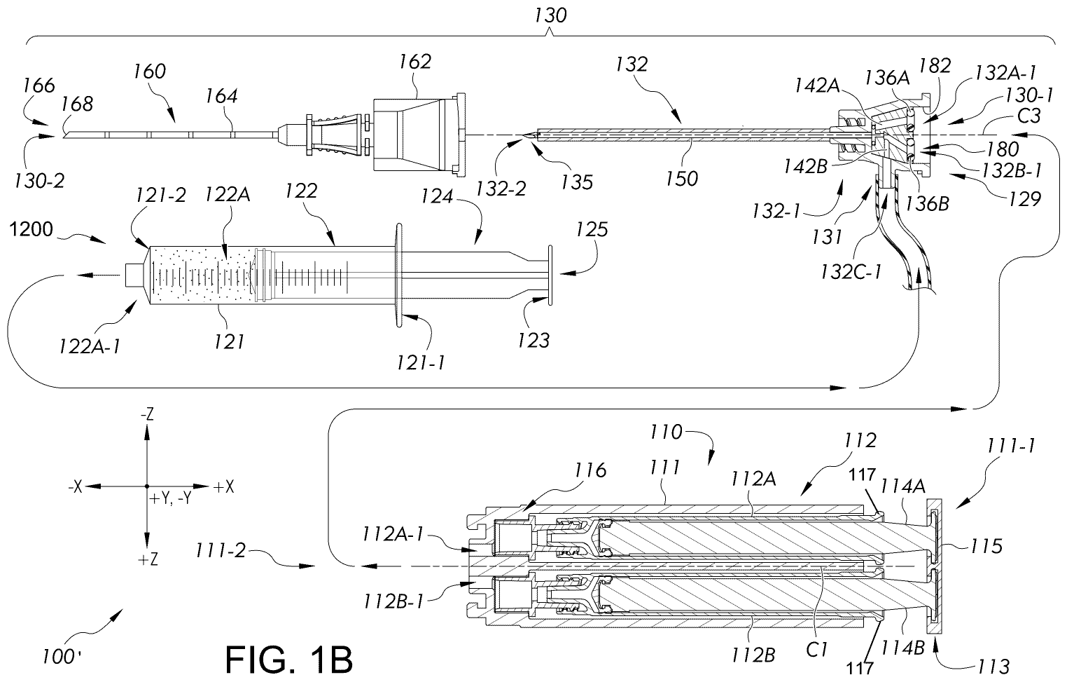

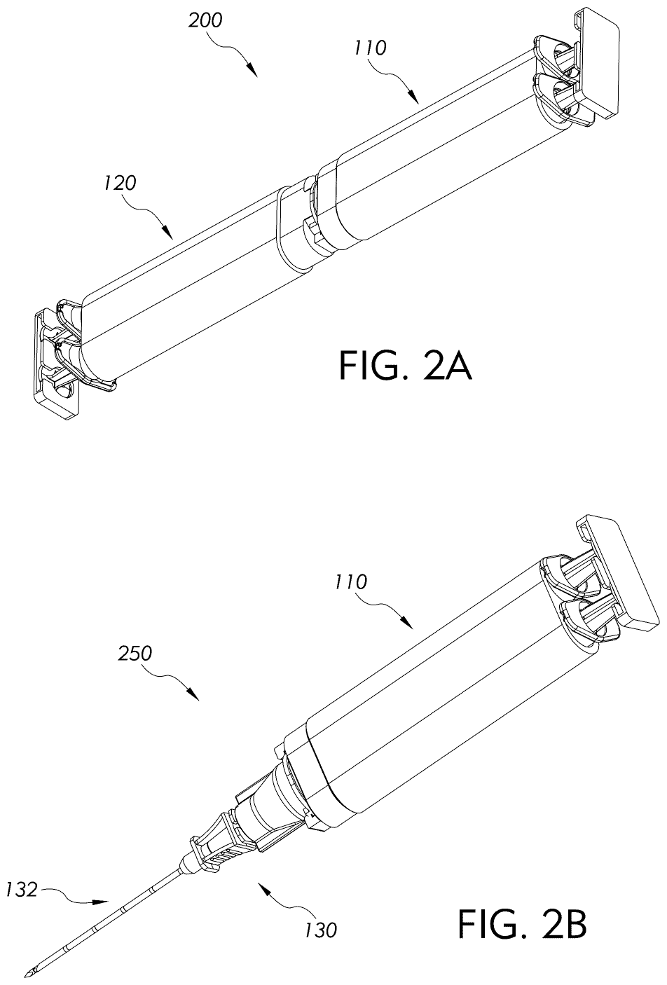

Turning now to the drawings, FIG. 1A depicts an illustrative component delivery system 100 according to various embodiments. The component delivery system 100, in accordance with an aspect of the present disclosure, may be for use in a lung access procedure to aid in preventing pneumothorax. The component delivery system 100 generally includes an applicator 110, a dual chamber mixing syringe 120, and/or an injection needle assembly 130. The various components for the component delivery system 100 are keyed via a connection system (e.g., a quarter turn connection system) such that the applicator 110 can be coupled to the dual chamber mixing syringe 120 for mixing components of a multi-component sealant, removed from the dual chamber mixing syringe 120, and coupled to the injection needle assembly 130 for further mixing and delivery of components, as indicated by the dashed lines in FIG. 1A and described herein. When coupled to the dual chamber mixing syringe 120, the combination of the dual chamber mixing syringe 120 and the applicator 110 may be referred to herein as a mixing apparatus 200, as depicted in FIG. 2A. When coupled to the injection needle assembly 130, the combination of the injection needle assembly 130 and the applicator 110 may be referred to herein as a delivery apparatus 250, as depicted in FIG. 2B.

FIG. 1B depicts an alternative illustrative component delivery system 100′ according to various embodiments. The component delivery system 100′, in accordance with at least one aspect of the present disclosure, may be for use in a delivery procedure whereby a multi-component material (e.g., a hydrogel) is delivered to a site that has been prepped via a hydro-dissection procedure. The component delivery system 100′ generally includes a dual chamber applicator 110, an auxiliary applicator 1200, and/or an injection needle assembly 130. The various components for the component delivery system 100′ are couplable together for the purposes of delivering a dual component mixture (e.g., a dual component hydrogel) and an additional material (e.g., saline or other hydro-dissection solution, or the like), as indicated by the dashed lines in FIG. 1B and described herein.

The auxiliary applicator 1200 generally includes a body 121 having a proximal end 121-1 and a distal end 121-2 spaced a distance apart from the proximal end 121-1. The body 121 also defines a syringe 122. The syringe 122 is configured to carry a material, such as a hydro-dissection solution (e.g., saline or another hydro-dissection solution), an anesthetic solution, and/or the like.

The syringe 122 includes an actuator 123 and a chamber 122A. The chamber 122A may be, for example, a cylindrical tube that is configured to carry the hydro-dissection solution, the anesthetic solution, and/or the like. The chamber 122A has an output port 122A-1.

In some aspects, the actuator 123 includes a piston 124 and a handle 125. The handle 125 is connected to the piston 124 at a proximal end thereof to facilitate movement of the piston 124 with the depression or retraction of the handle 125. The piston 124 is in the form of a plunger that is positioned in the chamber 122A proximal to the hydro-dissection solution, the anesthetic solution, and/or the like.

The output port 122A-1 of the chamber 122A may be arranged within the distal end 121-2 of the body 121 of the applicator 1200. The output port 122A-1 is generally a fluid output that is fluidly coupled to an auxiliary port 132C-1 of the hub 131 such that the fluid within the chamber 122A is deliverable via the auxiliary port 132C-1 of the hub 131. In some aspects, the output port 122A-1 may be couplable to the auxiliary port 132C-1 via tubing or the like, as depicted in FIG. 1B.

Referring to FIG. 2B, when the applicator 110 is coupled to the injection needle assembly 130, the delivery apparatus 250 therefrom has a configuration to facilitate delivery of a flowable multi-component material, such as a sealant, to an injection site. The multi-component material may be injected while the needle portion of the injection needle assembly 130 crosses the two layers of the pleura, (e.g., the parietal and visceral pleura). The injection needle assembly 130 is configured to puncture tissue and create an access path (e.g., a proposed biopsy tract) in the tissue at least through the pleura of the subject, and thus allows for the two layers of the pleura to be scaled (e.g., prior to a lung biopsy), by the curing of the multi-component material at the delivery site so that air cannot leak between the two layers and cause a pneumothorax. It is noted that the multi-component material also may be deposited in other regions of the access path, such as in the subcutaneous tissue and/or lung parenchyma.

Referring to FIGS. 1 and 2B, optionally, the delivery apparatus 250 may be used in conjunction with an introducer cannula 160. The introducer cannula 160 may facilitate withdrawal of the injection needle assembly 130 of the delivery apparatus 250 from a subject, while the introducer cannula 160 may remain in place to maintain the access path in the tissue to the site, such as for example, to receive and guide a second medical instrument, such as a biopsy device, to the site where the biopsy is to be performed.

Referring again to FIG. 1A, the applicator 110 generally includes a body 111 having a proximal end 111-1 and a distal end 111-2 spaced a distance apart from the proximal end 111-1. The body 111 also defines a pair of syringes 112. The applicator 110 is configured to separately carry each of a first component of the multi-component material and a second component of the multi-component material. In some instances, the multi-component material may be a sealant. The first component may include, for example, at least two N-hydroxysuccinimide (NHS) ester groups, and the second component may include, for example, at least two amine groups. For example, the first component may be a solution containing polyethylene glycol (PEG) succinimidyl succinate and the second component may be a solution containing albumin and/or polyethylenimine (PEI).

In other instances, the multi-component material may be a radiation spacer material. Referring to FIG. 1B, when the dual chamber applicator 110 is coupled to the injection needle assembly 130, the component delivery system 100′ facilitates delivery of flowable hydrogel precursor materials to an injection site. The hydrogel precursor materials may be injected when the needle portion of the injection needle assembly 130 is located in a target site, such as a space between the rectum and the prostate of a subject that is formed by prior injection of a hydro-dissection solution via the injection needle assembly 130.

The dual chamber applicator 110 of the component delivery system 100′ shown in FIG. 1B is configured to separately carry each of a first precursor material of the multi-component hydrogel and a second precursor material of the multi-component hydrogel. Illustrative examples of first precursor materials include, but are not limited to, albumin, polyethylenimine (PEI), an amine containing polyethylene glycol (PEG) or protein, or the like. Illustrative examples of second precursor materials include, but are not limited to, an N-hydroxysuccinimide (NHS) ester component such as PEG-(SS) 2, PEG-(SS) 4, PEG-(SS) 8, PEG-(SG) 4, PEG-(SG) 8, and/or the like. In some embodiments, molecular weights of the PEG components may range from about 2,000 to about 100,000.

Other multi-component uses are also contemplated and included within the scope of the present disclosure. In various instances, the first component and the second component are combined and mixed within a mixing chamber of the injection needle assembly 130, as will be described in greater detail herein.

The pair of syringes 112 includes an actuator 113, a first component chamber 112A, and a second component chamber 112B. The first component chamber 112A may be, for example, a cylindrical tube that is configured to carry the first component of the multi-component material. The first component chamber 112A has a first output port 112A-1 (e.g., a first component port). The second component chamber 112B also may be, for example, a cylindrical tube that is configured to carry the second component of the multi-component material. The second component chamber 112B has a second output port 112B-1 (e.g., a second component port). In some aspects, the first component chamber 112A and the second component chamber 112B are arranged in a substantially longitudinally parallel arrangement. While not depicted herein, in some aspects, the first component chamber 112A and the second component chamber 112B may each be nested component chambers such that each component chamber 112A, 112B contains two or more compartments therein for holding the various components of a multi-component material. For example, each component chamber 112A, 112B may be nested in a way that each contains two compartments so that there are four compartments total; each compartment holding a component of a multi-component material. Movement of the various components described herein may cause combination of the contents of each compartment within each component chamber to mix (e.g., to hydrate materials).

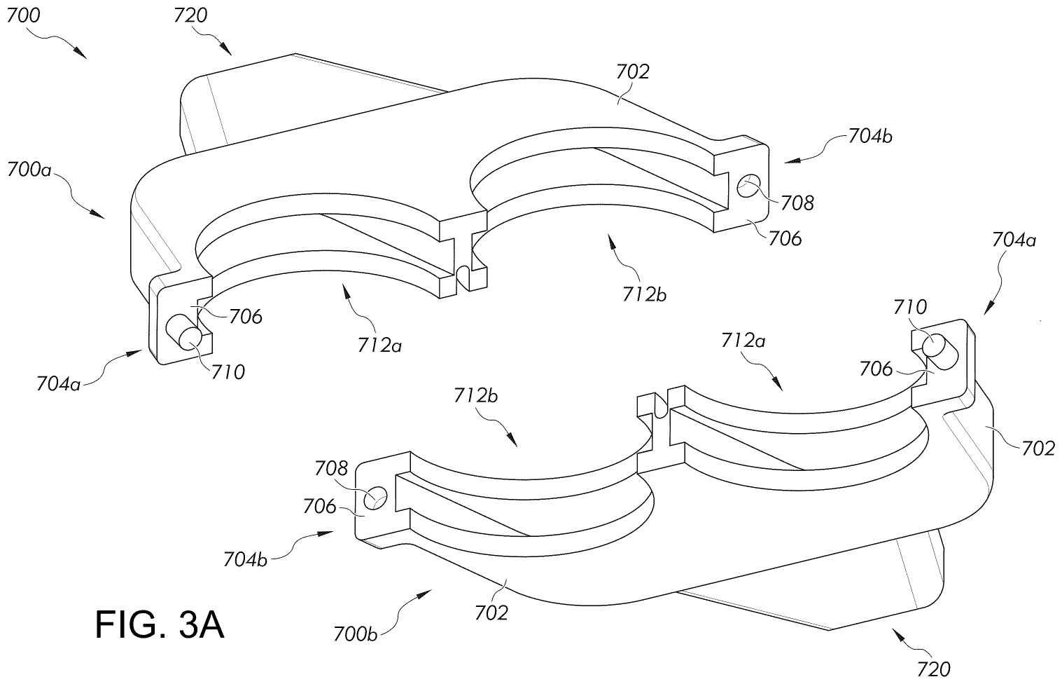

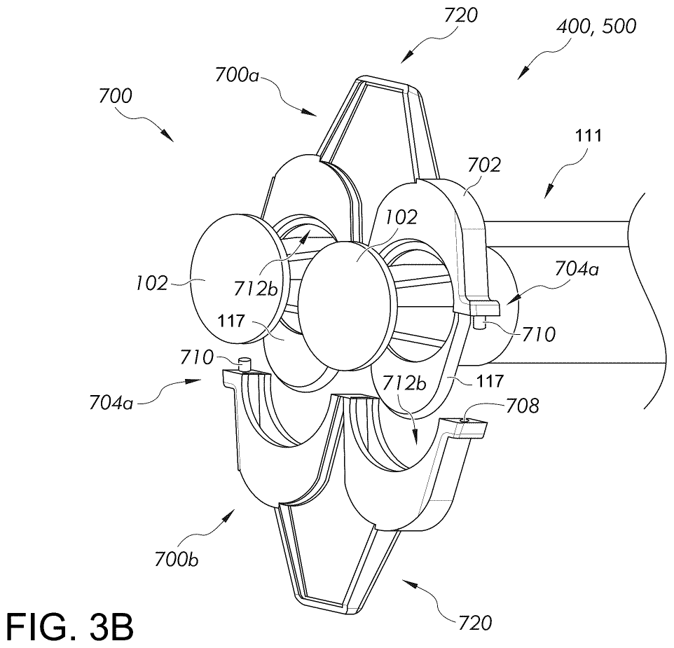

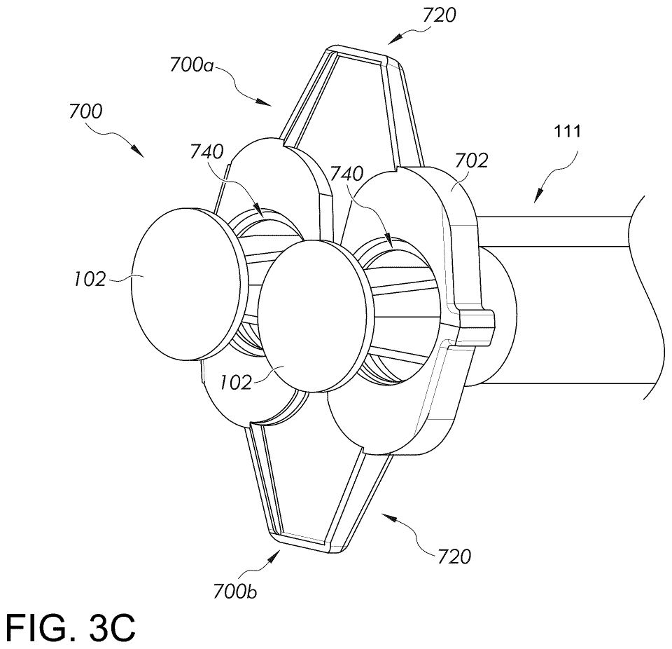

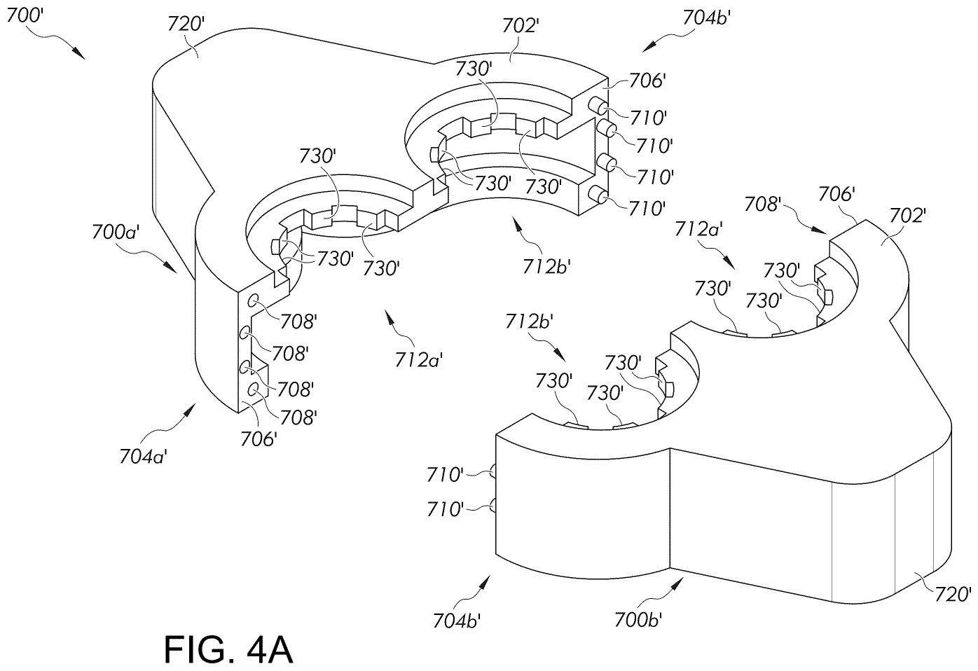

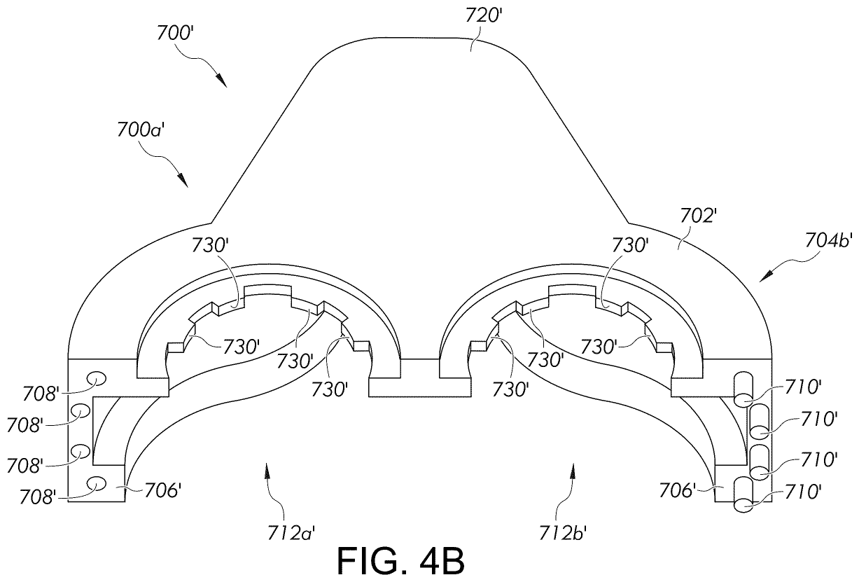

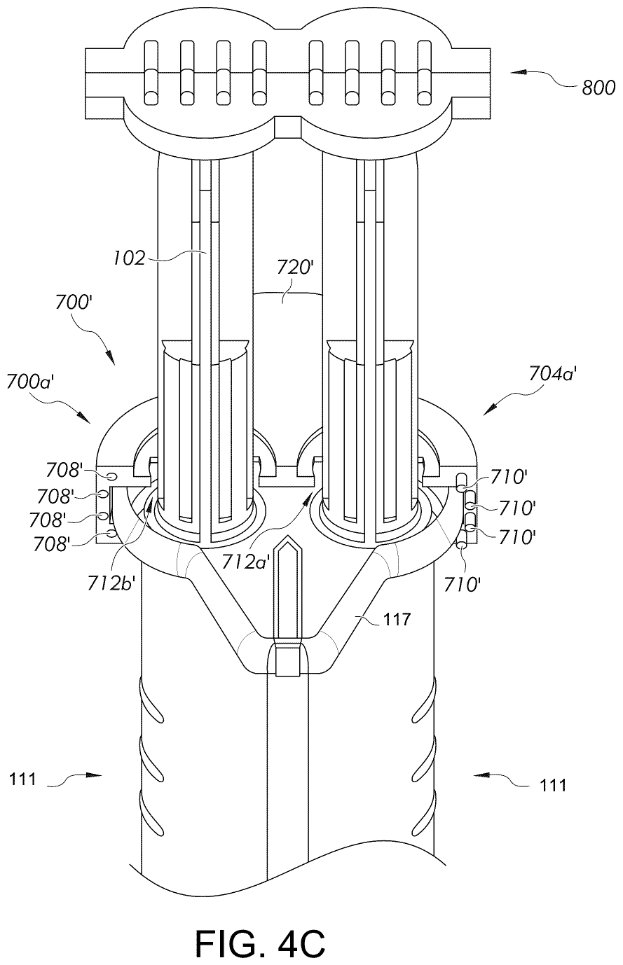

In various aspects, each of the first component chamber 112A and the second component chamber 112B may include a proximal end having a flange 117 (e.g., a flanged proximal end). As will be described herein with respect to FIGS. 3A-4C, for example, the flange 117 may provide a coupling area for coupling a two-piece alignment fixture.

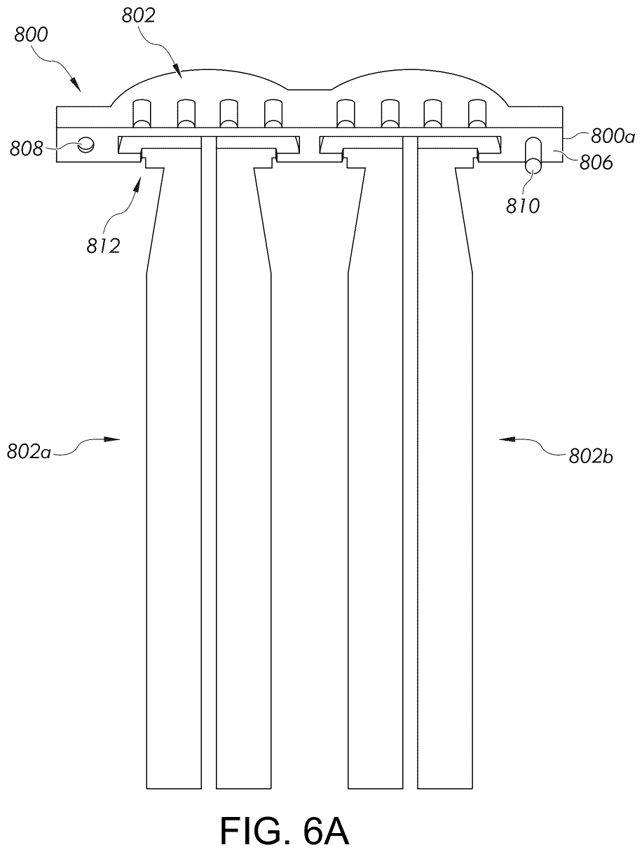

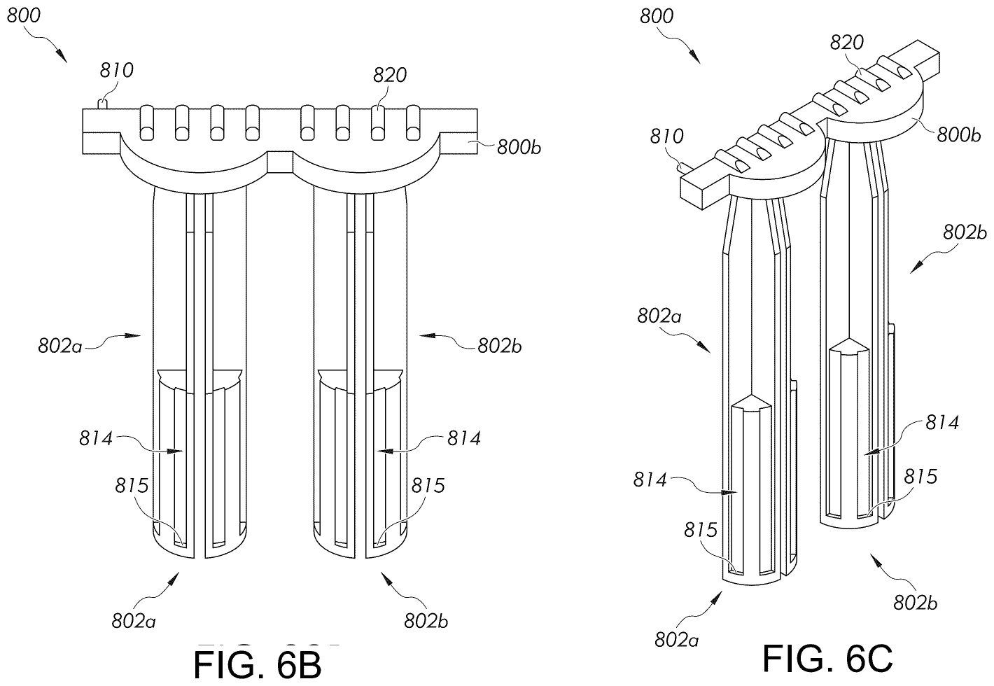

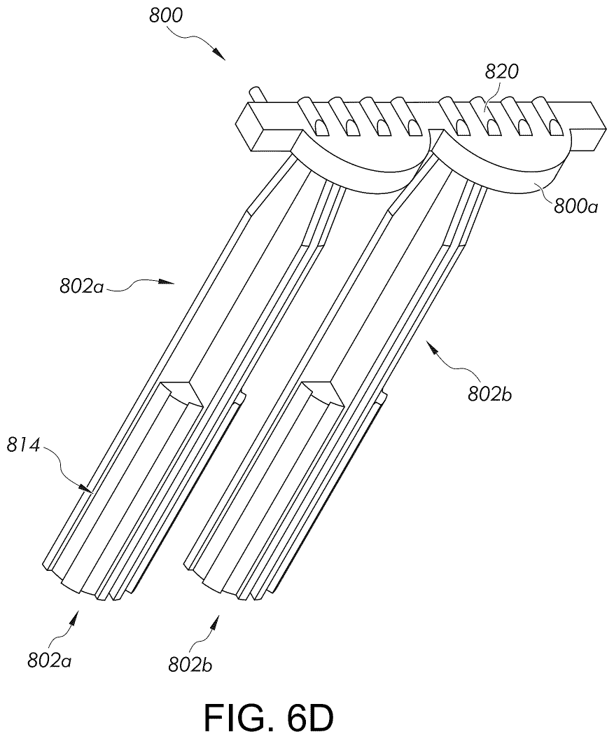

In some aspects, the actuator 113 includes a first piston 114A, a second piston 114B, and a handle 115. The handle 115 is in the form of a link member that perpendicularly extends between, and is connected to, each of the first piston 114A and the second piston 114B to facilitate simultaneous movement of the first piston 114A and the second piston 114B with the depression or retraction of the handle 115. The handle 115 is formed from the two-piece piston insert 800 (FIGS. 6A-6D), as described in greater detail herein. For example, the handle 115 couples to a first thumb press 114A-1 of the first piston 114A and second thumb press 114B-1 of the second piston 114B. As will be described in greater detail herein, movement of the handle 115 by a user is facilitated by a two-piece alignment fixture coupled to the flange 117 as well as the two piece piston insert coupled thereto. The first piston 114A is in the form of a plunger that is positioned in the first component chamber 112A proximal to the first sealant component, and the second piston 114B is in the form of a plunger that is positioned in the second component chamber 112B proximal to the second sealant component.

The first output port 112A-1 of the first component chamber 112A and the second output port 112B-1 of the second component chamber 112B may be arranged within the distal end 111-2 of the body 111 of the applicator 110. The first output port 112A-1 and the second output port 112B-1 are generally fluid outputs that are aligned with other ports of other components as described herein such that the first and second components can be dispensed from and/or received within the respective component chambers 112A, 112B. In some aspects, the first output port 112A-1 may be concentrically aligned with the first component chamber 112A and the second output port 112B-1 may be concentrically aligned with the second component chamber 112B. However, in other aspects, such as the aspect depicted in FIGS. 1A and 1B, the first output port 112A-1 may be located radially inward of a central area of the first component chamber 112A and the second output port 112B-1 may be located radially inward of a central area of the second component chamber 112B such that the first output port 112A-1 and the second output port 112B-1 are as close as possible to a center axis C1 of the body 111 of the applicator 110 to facilitate alignment with the other components of the component delivery system 100 described herein.

The body 111 of the applicator 110 may have one or more coupling components for coupling the body 111 to various other devices, such as, for example, the injection needle assembly 130, a dual chamber mixing syringe (not depicted), and/or the like. While the present disclosure is not related to any specific coupling components, illustrative coupling components that may be used include a quarter turn connector or any other suitable connector. For example, the body 111 of the applicator 110 may include a connector 300 integrated with the distal end 111-2 of the applicator 110. More specifically, as depicted in FIGS. 1A and 1B, the various components of the quarter turn connector 300 are integrated with the body 111 such that the quarter turn connector 300 and the body 111 are a single monolithic piece. However, it should be understood that this is merely illustrative and the various components of the quarter turn connector 300 may be separate pieces that are permanently or semi-permanently joined with the body 111 of the applicator 110 (e.g., permanently or semi-permanently joined with a distal coupling piece 116 of the applicator 110).

The quarter turn connector 300 is generally located at the distal end 111-2 of the body 111 of the applicator such that various components of the quarter turn connector 300 are positioned adjacent to first output port 112A-1 and the second output port 112B-1. As will be described herein, the quarter turn connector 300 is generally shaped and sized to releasably interlock with a corresponding quarter turn connector 400 of the dual chamber mixing syringe 120, and/or with a corresponding quarter turn connector 450 of the injection needle assembly 130. As will be described in greater detail herein, when the injection needle assembly 130 or the dual chamber mixing syringe 120 is coupled to the applicator 110 via the quarter turn connectors 300, 400, 450 thereof, the various ports thereof are aligned and sealed with the first output port 112A-1 and the second output port 112B-1 of the applicator 110.

Still referring to FIG. 1A, the quarter turn connector 300 of the applicator 110 includes a protrusion 302 extending distally (e.g., in the −x direction of the coordinate axes of FIG. 1) from the distal end 111-2 of the applicator 110, a channel disposed within the distal end 111-2 of the applicator 110 along a periphery of the protrusion 302, and a pair of bayonet coupling members (e.g., a first coupling member 306A and a second coupling member 306B) disposed radially outward of the protrusion 302 and the channel 304. As shown in FIG. 1, the first output port 112A-1 and the second output port 112B-1 are disposed within the protrusion 302. That is, the openings into the first component chamber 112A and the second component chamber 112B are located on the protrusion 302.

The protrusion 302 is generally shaped and sized to correspond to a recess formed in the injection needle assembly 130 and the dual chamber mixing syringe 120 (or other component to be coupled). In various instances, the protrusion 302 is circular. The protrusion 302 may generally be disposed in or around a central area of the distal end 111-2 of the body 111. In some embodiments, the protrusion 302 may be concentric with the body such that the center axis C1 of the body 111 extends through a center of the protrusion 302. The distance that the protrusion 302 extends away from the distal end 111-2 of the body is generally a distance that corresponds to a depth of the recess formed in the injection needle assembly 130 and the dual chamber mixing syringe 120 (or other component to be coupled) such that the protrusion 302 can be completely inserted therein, but is otherwise not limited by the present disclosure.

Referring again to FIG. 1, the first coupling member 306A and the second coupling member 306B each extend from the distal end 111-2 of the body 111 of the applicator 110 and are generally shaped and sized to retain the dual chamber mixing syringe 120 or the injection needle assembly 130 when coupled to the applicator 110. Each of the first coupling member 306A and the second coupling member 306B may be a bayonet style coupling member, an L-beam coupling member, or the like. For example, as particularly depicted in FIG. 1, the first coupling member 306A extends distally at a particular distance from the distal end 111-2 of the body 111, turns about 90 degrees, and extends inward toward the center axis C1 of the body 111, resulting in a first extension piece 306A-1 that extends in a direction generally coplanar with the center axis C1 of the body 111 (e.g., along the x-axis of the coordinate axes of FIG. 1) and a second extension piece 306A-2 that extends in a direction that is generally perpendicular to the center axis C1 of the body (e.g., along the z-axis of the coordinate axes of FIG. 1), thereby defining a pocket 307A between the distal end 111-2 of the body 111 and the second extension piece 306A-2. Similarly, the second coupling member 306B extends distally at a particular distance from the distal end 111-2 of the body 111, turns about 90 degrees, and extends inward toward the center axis C1 of the body 111, resulting in a first extension piece 306B-1 that extends in a direction generally coplanar with the center axis C1 of the body 111 (e.g., along the x-axis of the coordinate axes of FIG. 1) and a second extension piece 306B-2 that extends in a direction that is generally perpendicular to the center axis C1 of the body 111 (e.g., along the z-axis of the coordinate axes of FIG. 1), thereby defining a pocket 307B between the distal end 111-2 of the body 111 and the second extension piece 306B-2.

As depicted in FIG. 1, the first coupling member 306A and the second coupling member 306B are located opposite one another, radially outward of the protrusion 302 and the channel 304. However, this is merely illustrative, and other locations and spacing are contemplated and included within the scope of the present disclosure. Further, while the present aspect includes a pair of coupling members (e.g., the first coupling member 306A and the second coupling member 306B), this is also merely illustrative. That is, other amounts of coupling members are also contemplated and included within the scope of the present disclosure.

Still referring to FIGS. 1A and 1B, the injection needle assembly 130 has a proximal end 130-1 that extends proximally (in the +x direction of the coordinate axes of FIG. 1) and a distal end 130-2 that extends distally (e.g., in the −x direction of the coordinate axes of FIG. 1). The injection needle assembly 130 generally includes a hub 131 and an elongate hollow stylet 132 that extends distally (e.g., in the −x direction of the coordinate axes of FIG. 1) from hub 131. The elongate hollow stylet 132 has a proximal end 132-1 and a distal end 132-2. The hub 131 is fixedly attached, e.g., through overmolding, adhesive and/or pressed fit, to the proximal end 132-1 of the elongate hollow stylet 132. The hub 131 includes a plurality of input ports (e.g., a first input port 132A-1 and a second input port 132B-1) of the injection needle assembly 130. The hub 131 is configured for removable connection to the applicator 110 via the corresponding quarter turn connector 450 such that, when connected, the first input port 132A-1 of the injection needle assembly 130 is aligned and sealed with the first output port 112A-1 of the applicator 110 and the second input port 132B-1 of the injection needle assembly 130 is aligned and sealed with the second output port 112B-1 of the applicator 110, as described in greater detail herein.

The elongate hollow stylet 132 of injection needle assembly 130 is configured to facilitate fluid communication with the plurality of output ports 112A-1, 112B-1 of the applicator 110 so as to receive the two components of the multi-component material from the applicator 110 and direct the two components to the distal end 132-2 thereof for mixing and delivery. The closed distal end 133 of the elongate hollow stylet 132 may be, for example, a closed stylet needle tip 135.

The elongate hollow stylet 132 may be constructed, for example, by an elongate cannula 150 being fixedly connected to the closed stylet needle tip 135, with the plurality of side ports 134 being located in the elongate cannula 150 in a distal chamber 151 thereof that is immediately proximal to the closed stylet needle tip 135. More particularly, the elongate cannula 150 of the elongate hollow stylet 132 defines an outer side wall 152 that surrounds an outer lumen 153 of the elongate hollow stylet 132. In addition, located within the outer lumen 153 of the elongate cannula 150 is an inner side wall 154 that surrounds an inner lumen 155, the inner lumen being concentric with the outer lumen such that a passageway is defined between the inner side wall 154 and the outer side wall 152. Furthermore, a distal opening 153-1 of the outer lumen 153 and a distal opening 155-1 of the inner lumen 155 are open to the distal chamber 151 of the elongate hollow stylet 132.

Referring to FIGS. 3A-3C, an illustrative two-piece alignment fixture 700 is depicted, which may be particularly useful in the dual barrel embodiments described herein, such as with respect to FIG. 1, though it is contemplated that the two-piece alignment fixture 700 may be applicable to any barrel design and need not necessarily include all of the features described herein. Additionally, the fixture may also provide a more comfortable and intuitive grip for a user during a hydration step.

In general, the two-piece alignment fixture 700 has a body 702 comprised of two parts: a first section 700a and a second section 700b. The two parts of the two-piece alignment fixture 700 may be substantially identical and/or mirror images of one another; as such, the various components for each will be numbered the same and described collectively for purposes of brevity.

The body 702 extends between a first end 704a and a second end 704b, and has a face 706. The face 706 includes various components that allow the two-piece alignment fixture 700 to be coupled to an applicator, such as the applicator 110 (FIG. 1). In various instances, the applicator includes a dual mixing syringe assembly. Particularly, the body 111 of the applicator 110, such as the first component chamber 112A and the second component chamber 112B, may each include a flange 117, see FIG. 1. The flanges 117 may be separate from one another. The two-piece alignment fixtures may extend around the flanges 117 such that the respective flanges 117 of each of the first component chamber 112A and the second component chamber 112B are coupled to one another. In some embodiments, the flanges 117 may already be coupled to one another and the fixture may provide an additional gripping surface, for improved control and/or use.

To assist in coupling the two sections 700a, 700b together, the face 706 of the first section 700a and the second section 700b, such as at the first end 704a of the body 702 includes a protrusion 710, such as a detent, a knob, a post, or the like. The face 706 of the first section 700a and the second section 700b, such as at the second end 704b of the body 702 may include a mating recess 708. The protrusion 710 and the recess 708 may be complementary with respect to each other so that the protrusion 710 of the first section 700a fits within the recess 708 of the second section 700b, and the protrusion 710 of the second section 700b fits within the recess 708 of the first section 700a. In addition, the protrusion 710 and the recess 708 may be shaped and sized so that the first and second sections 700a, 700b are held together when joined. For example, the protrusion 710 may be flared, have retention pieces, or the like that provides a friction, or otherwise tight, fit within the recess 708 when the first and second sections 700a, 700b are joined together.

To assist in in mounting the two-piece alignment fixture 700 to the flanges 117 noted above, the face 706 may include a recess and/or a channel 712a, 712b that is shaped and sized to correspond to the flanges 117 of the component chambers 112A, 112B so that the flanges 117 are received within the recess and/or channel 712a, 712b when the first section 700a and the second section 700b are joined together around the flanges 119, as particularly depicted in FIG. 3C. As shown in FIG. 3C, tabs 720 extending from the body 702 of the two-piece alignment fixture 700 provide a location for a user to hold the assembly while depressing or retracting plungers 102, similar in many respects to the first piston 114A and the second piston 114B shown in FIG. 1. When assembled, or otherwise in an assembled state, the two-piece alignment fixture 700 provides an opening 740 therethrough for advancement and/or retraction of the plungers 102.

Referring to FIGS. 4A-4C, another two-piece alignment fixture 700′ is depicted, which may be used in place of the two-piece alignment fixture 700 described above or as otherwise described. The two-piece alignment fixture 700′ has a body 702′ comprised of two parts: a first section 700a′ and a second section 700b′. The two parts of the two-piece alignment fixture 700′ may be substantially identical and/or mirror images of one another; as such, the various components for each will be numbered the same for purposes of brevity.

The body 702′ extends between a first end 704a′ and a second end 704b′, and has a face 706′. The face 706′ includes various components that allow the two-piece alignment fixture 700′ to be coupled to any suitable applicator, such as applicator 110 (FIG. 1), particularly the flanges thereof, such as a proximal flange 117 of the body 111 of the component chambers 112A, 112B, as described herein. For example, the face 706 at the first end 704a′ of the body 702′ includes a protrusion 710′, such as a detent, a knob, a post, or the like. In another example, the face 706′ at the second end 704b′ of the body 702′ includes a recess 708′. The protrusion 710′ and the recess 708′ may be complementary with respect to each other so that the protrusion 710′ of the first section 700a′ fits within the recess 708′ of the second section 700b′, and the protrusion 710′ of the second section 700b′ fits within the recess 708′ of the first section 700a′. In addition, the protrusion 710′ and the recess 708′ may be shaped and sized so that the first and second sections 700a′, 700b′ are held together when joined. For example, the protrusion 710′ may be flared, have retention pieces, or the like that provides a friction, or otherwise tight, fit within the recess 708′ when the first and second sections 700a′, 700b′ are joined together. There may be any number of recesses and/or protrusions incorporated into the two-piece alignment fixture 700′ to couple the first and second sections 700a′, 700b′ to one another, such as a plurality thereof.

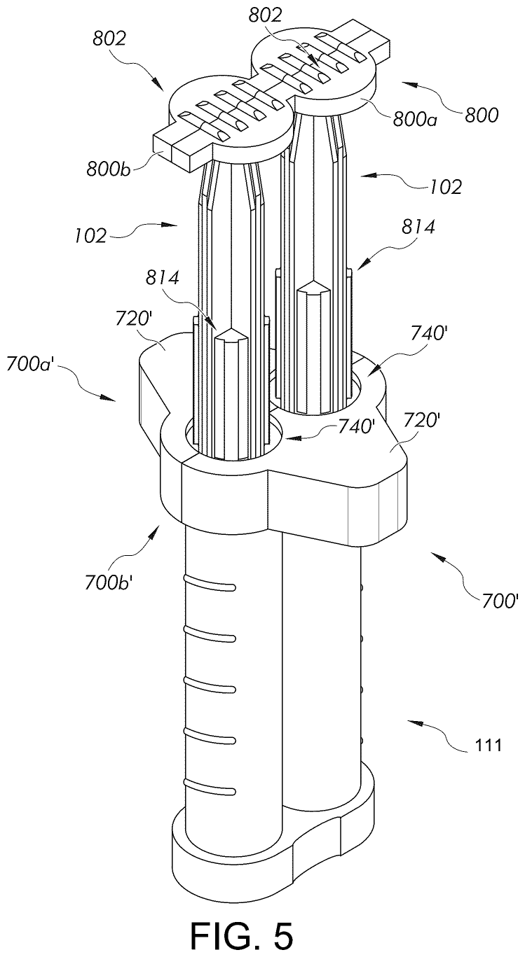

The face 706′ further includes a recess and/or a channel 712a′, 712b′ that is shaped and sized to correspond to the flange(s) 117 of any suitable applicator, such as applicator 110 (FIG. 1), so that the flange(s) 117 are received within the recess and/or channel 712a′, 712b′ when the first section 700a′ and the second section 700b′ are joined together around the flange 117, as particularly depicted in FIGS. 4C-5. It is noted that FIG. 4C illustrates an embodiment where there is a common flange 117 connecting barrel bodies 106, though in embodiments, the flanges 117 may be separate. As shown in FIG. 5, tabs 720′ extending from the body 702′ of the two-piece alignment fixture 700′ provide a location for a user to hold the mixing syringe assembly while depressing or retracting the plungers 102 of the respective first and second mixing syringe subassemblies.

As in the embodiment above, when assembled, the two-piece alignment fixture 700′ provides an opening 740′ therethrough for advancement and/or retraction of the plungers 102. In the depicted embodiment, the two-piece alignment fixture 700′ may include protrusions 730′ or the like that extend radially into the opening 740′. Such protrusions 730′ are shaped and sized to fit within a corresponding channel of a two-piece piston insert 800, as described in greater detail herein.

Referring now to FIGS. 4C-6D and 8, an illustrative two-piece piston insert 800 is depicted. As shown specifically in FIGS. 5-6D and 8, the two-piece piston insert 800 has a body 802 comprised of two parts: a first section 800a and a second section 800b. The two parts of the two-piece piston insert 800 may be substantially identical and/or mirror images of one another; as such, the various components for each will be numbered the same for purposes of brevity. The two-piece piston insert 800 may act as a plunger connector connecting the plunger 102 of the first mixing syringe subassembly to the plunger 102 of the second mixing syringe subassembly, such that movement of one of the plungers 102 moves the other of the plungers 102 (e.g., the plungers 102 move in concert). Further, a force can be applied to the two-piece piston insert 800 in any area thereof (e.g., the area located between the two plungers 102) to cause movement of both plungers 102.

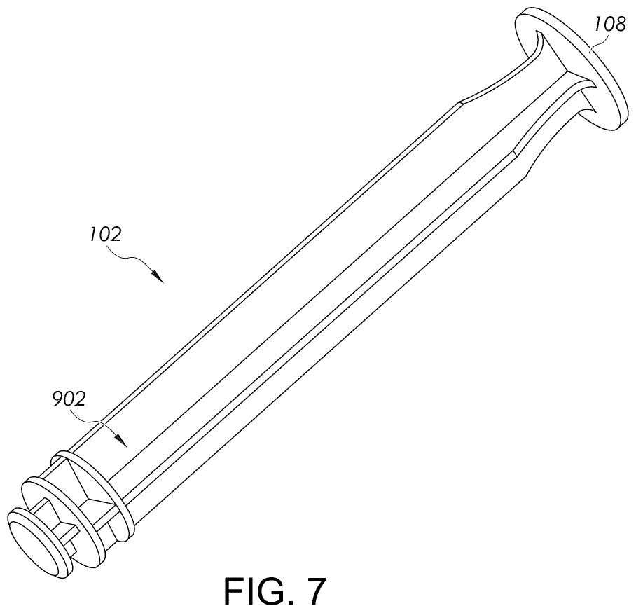

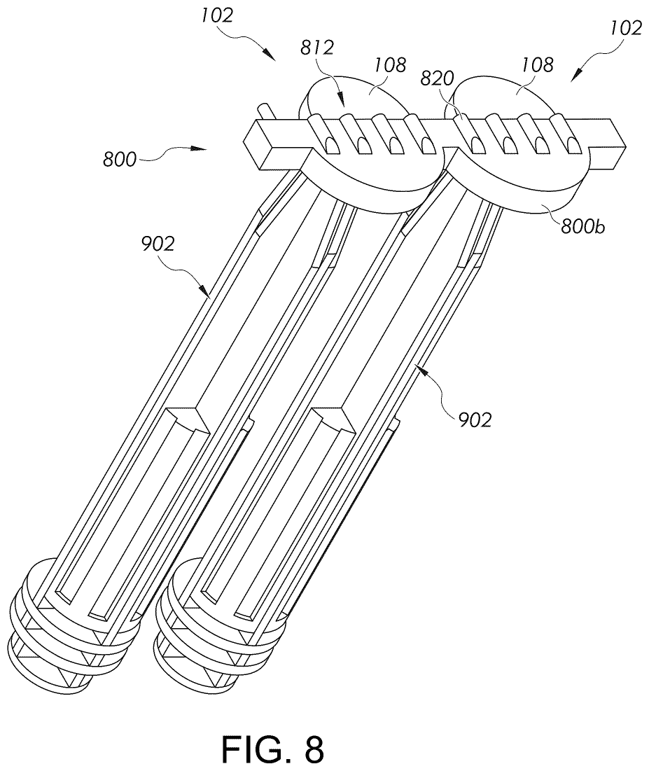

The body 802 extends between a first end and a second end, and has a face 806. A pair of extension pieces 802a, 802b extend distally from the body 802 and are molded to fit within grooves 902 of a corresponding plunger 102, as particularly shown in FIGS. 7 and 8. Accordingly, the pair of extension pieces 802a, 802b may be separated from one another via a channel. The face 806 includes various components that allow the two-piece piston insert 800 to be coupled to the plungers 102. For example, the face 806 at the first end of the body 802 includes a protrusion 810, such as a detent, a knob, a post, or the like. In another example, the face 806 at the second end of the body 802 includes a recess 808. The protrusion 810 and the recess 808 may be complementary with respect to each other so that the protrusion 810 of the first section 800a fits within the recess 808 of the second section 800b, and the protrusion 810 of the second section 800b fits within the recess 808 of the first section 800a. In addition, the protrusion 810 and the recess 808 may be shaped and sized so that the first and second sections 800a, 800b are held together when joined. For example, the protrusion 810 may be flared, have retention pieces, or the like that provides a tight fit within the recess 808 when the first and second sections 800a, 800b are joined together.

Other components of the face 806 include a recess and/or a channel 812 that is shaped and sized to correspond to one or more flanges 108 of the plunger 102 so that the one or more flanges 108 are received within the recess and/or channel 812 when the first section 800a and the second section 800b are joined together around the plunger 102, as particularly depicted in FIG. 5. The resulting combination of the two-piece piston insert 800 and the plungers 102 is depicted in FIG. 5. In addition, as shown in FIGS. 5 and 8, surface features 820 extending from the body 802 of the two-piece piston insert 800 provide a location for a user to manipulate the pistons 114A, 114B. The surface features 820 may include an array of raised bumps or ridges, though other surface features are contemplated and possible.

It should be understood that the two-piece piston insert 800 is an attachment device for a plunger 102 that provides increased surface area of the combined insert/plunger to be in contact with the inner wall of the body 111, thereby providing increased stability. For example and as depicted, the two-piece piston insert 800 traces down the stem of the plunger 102 so as to be inserted in to the grooves 902 of the plunger 102. In embodiments, at approximately a half way, or otherwise mid, point of a length of the insert/plunger 800/102, a radius is provided via the combination of the insert/plunger 102 matching that of the syringe barrel inner diameter (however this radius could be produced at any suitable location). The increased radius may assist in providing additional stability to the plungers 102. For example, as the plungers 102 are most distal from an end face of the body 111, the two-piece piston insert 800 provides a stabilizer attachment that reduces perpendicular motion (from respect to the line of plunger 102 travel). This allows for a smoother deployment of the contents within the body 111.

It is noted that in some embodiments, the two-piece piston insert 800 may not be included, instead features may be directly molded into the plunger 102, to minimize plunger wobbling during use, for example.

In embodiments, the protrusions 730′ of the two-piece alignment fixture 700′ may engage with the two-piece piston insert 800. For example, the pair of extension pieces 802a, 802b may include longitudinal grooves 814 corresponding to the protrusions 730′ of the two-piece alignment fixture 700′. The longitudinal grooves 814 may end in a stop wall 815. When the plungers 102 and two-piece piston insert 800 are pulled proximally, or otherwise retracted, the stop wall 815 may engage with the protrusions 730′ and prevent complete withdrawal of the plunger 102 and the two-piece piston insert 800 from the respective component chambers 112A, 112B and two-piece alignment fixture 700′. Stated another way, the plungers 102 are able to freely move within the component chambers 112A, 112B until the plungers 102 are retracted to a particular location where the stop wall 815 engages with the protrusions 730′ to prevent complete withdrawal of the plunger 102 and the two-piece piston insert 800 from the respective component chambers 112A, 112B. Accordingly, the plunger 102 and two-piece piston insert 800 may be locked into the assembly thereby preventing accidental disassembly of the plunger 102 from the barrel body 106. In some embodiments, such as illustrated in FIG. 6D, a stop wall may be omitted.

While various instances herein describe the alignment fixture and the piston insert each as including two pieces, it is envisioned that the alignment fixture can be a single, integral piece and/or the piston insert can be a single, integral piece. Moreover, in various instances, the first piston and the second piston can be integrally formed with one another, or otherwise connected and/or coupled as a single component.

It should now be understood that the present disclosure relates to both a two-piece clip-in fixture which fixes around the wings of a dual chamber syringe and a two-piece piston insert that couples to the pistons. This allows for the small syringes to be bound together and enables a homogeneous distribution of pressure when hydrating the raw chemistry in the main barrel. The present disclosure also adds a more comfortable and intuitive grip for the user during a hydration step and is also designed to avoid accidental removal of piston(s) from the respective barrels during the hydration step. This homogenous distribution of pressure allows for a more ergonomic device operation and prevents the user from having to move from one syringe to the other in order to expel all of the hydration agent. The devices and systems described herein also aid as a better finger grip or provide better tactility during a hydration step compared to conventional designs.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A component delivery system, comprising:
a dual chamber applicator, comprising:
a body, comprising:
a first component chamber; and
a second component chamber;
a first piston slidably engaged with the first component chamber; and
a second piston slidably engaged with the second component chamber;
an alignment fixture removably coupled to the body of the dual chamber applicator; and
a piston insert removably couplable to the first piston and the second piston to connect the first piston to the second piston.

2. The component delivery system of claim 1, wherein the alignment fixture comprises:
a first section comprising a first face; and
a second section removably coupled to the first section in an assembled state, the second section comprising a second face.

3. The component delivery system of claim 2, wherein the first face comprises a projection and the second face comprises a mating recess sized to receive the projection therein.

4. The component delivery system of claim 2, wherein the alignment fixture defines an opening in the assembled state to receive the first piston therethrough.

5. The component delivery system of claim 4, wherein the alignment fixture further comprises a protrusion extending radially into the opening, wherein the protrusion is sized to fit within a channel defined in the piston insert.

6. The component delivery system of claim 1, wherein the first piston and the second piston are integrally formed.

7. The component delivery system of claim 2, wherein the dual chamber applicator further comprises a flange defined on a proximal end thereof, wherein the first face of the alignment fixture comprises a channel defined therein, and wherein the channel is sized to receive the flange of the dual chamber applicator therein.

8. The component delivery system of claim 1, wherein the piston insert comprises:
a first extension piece removably coupled to the first piston to prevent removal of the first piston from the first component chamber; and
a second extension piece removably coupled to the second piston to prevent removal of the second piston from the second component chamber.

9. A component delivery system, comprising:
an applicator comprising two chambers separate from one another, each chamber comprising at least one output port on a distal end thereof for receiving corresponding pistons;
a two-piece alignment fixture removably couplable to the applicator; and
a two-piece piston insert removably couplable to the pistons, the two-piece piston insert engagable with the two-piece alignment fixture to prevent removal of the pistons from the two chambers.

10. The component delivery system of claim 9, wherein the two-piece alignment fixture comprises one or more tabs extending from a body to provide a location for a user to hold the component delivery system.

11. The component delivery system of claim 9, wherein the two-piece piston insert comprises a pair of extension pieces sized to extend into the two chambers.

12. The component delivery system of claim 11, wherein the two-piece alignment fixture defines an opening in an assembled state to receive the pair of extension pieces of the two-piece piston insert therethrough.

13. The component delivery system of claim 12, wherein the pair of extension pieces comprises longitudinal grooves ending in a stop wall.

14. The component delivery system of claim 13, wherein the two-piece alignment fixture comprises protrusions extending radially into the opening, wherein the protrusions are sized to fit within the longitudinal grooves defined in the two-piece piston insert.

15. The component delivery system of claim 14, wherein the protrusions engage the stop wall of the pair of extension pieces to prevent a complete withdrawal of the pistons from the two chambers.

16. The component delivery system of claim 9, wherein the two-piece piston insert comprises a body comprising a surface feature extending therefrom.

17. The component delivery system of claim 16, wherein the surface feature comprises an array of raised bumps.

18. An alignment fixture for coupling to an applicator, the alignment fixture comprising:
a body, comprising:
a first section;
a second section removably coupled to the first section in an assembled state, the body defining an opening in the assembled state to receive a plunger of the applicator therethrough; and
a face comprising a feature to facilitate coupling of the alignment fixture to the applicator.

19. The alignment fixture of claim 18, wherein the feature comprises a protrusion extending from the face.

20. The alignment fixture of claim 18, wherein the body further comprises a tab extending from the body to provide a location for a user to hold the alignment fixture.
